(19) United States
(12) Patent Application Publication
(10) Pub. No.: US 2014/0277136 A1
(43) Pub. Date: Sep. 18, 2014

(54) PORTABLE WIRELESS POINT-OF-SALE VENDING MACHINE

(71) Applicant: Spencer Mason STEIN, Del Mar, CA (US)

(72) Inventor: Spencer Mason STEIN, Del Mar, CA (US)

(21) Appl. No.: 13/797,236

(22) Filed: Mar. 12, 2013

(51) Int. Cl. G06Q 10/08; A61F 5/00

(52) U.S. Cl. CPC G06Q 10/087; A61F 5/00
USPC 606/240; 700/236

(57) ABSTRACT

At least one embodiment of the invention describes a portable wireless point-of-sale vending machine that is easily moved and carried by a user. The vending machine includes an outer surface and at least one inner portion enclosed by the outer surface. The one or more inner portions include one or more receptacles, where each of the receptacles contains one or more reusable and interchangeable mechanical items. The vending machine may also include a wireless transceiver and embedded or removable computer that communicate with at least one third party in a bidirectional manner. The mechanical items are dispensed out of the outer surface as needed and the entire portable wireless point-of-sale vending machine may also be structurally used as an exercise device, integrated service device, carrying device, baby carriage or a physical therapy device.

### BACKGROUND OF THE INVENTION

1. Field of the Invention

One or more embodiments of the invention are related to wireless vending machines, specifically portable wireless point-of-sale vending machine. More particularly, but not by way of limitation, one or more embodiments of the invention enable a user to use the vending machine to dispense and pay for the use and/or cleaning of at least one reusable and interchangeable mechanical item, as well as to use the vending machine itself as an exercise device, integrated service device, carrying device, baby carriage or a physical therapy device.

2. Description of the Related Art

Generally, vending machines involve the dispensation of an item of a user's choice for a set price. Vending machines hold a wide range of items from bottled beverages, packaged snacks, newspapers, sanitary needs, electronics, DVDs, tickets, books and many others. Most often, the vending machines are in a stationary non-portable location on either the ground or wall-mounted. Traditional vending machines also include locking and unlocking elements, as well as different types of payment accepting structures that allow one or more users to purchase vending machine items to be dispensed on one-time basis.

Many different vending machines have been used in office buildings, hotels, retail stores, restaurants, bathrooms and other locations easily accessible to the public, and equally accessible to more than one user at once. Some vending machines include computer-enabled elements that use RFID technology and sensors for communicating inventory supply, usage and consumption.

For example, United States Patent Publication 20100096293 to Libermann, entitled “Electronic Medication Dispensing System and Related Utilities”, discloses a medication dispensing system for dispensing pills and medical ointments in the form of tubes. The dispensing system may be carried and/or moved by a user for storage and consumption of the medical items. Libermann's dispensing system, for example, also includes a communication system for communicating with one or more third parties such as healthcare providers for receiving information about medications, products, services and advertising.

United States Patent Publication 20070073113 to Squilla, John R. et al. entitled “Providing Medical Services At A Kiosk” discloses a stationary medical kiosk for providing medical services, for example medical tests. The medical kiosk, for example, dispenses a test kit for the user to perform the necessary test, after which the test kits are retrieved and analyzed to provide the user with the examination results.

Traditionally, the vending machines themselves are not equipped and structured to be portable and function as integrated service or physical therapy dispensing machines as well as function as exercise equipment such as a physical therapy postural alignment device, or carrying device or baby carriage for example. Furthermore, traditional vending machines and kiosks are not implemented as portable devices to be rented out to a single user for a limited time that is in bidirectional communication with one or more third parties to control usage, consumption, inventory, invoices and location services while the vending machine or kiosk is in a non-stationary position. In addition, traditional vending machines are not equipped with embedded or removable computer elements that enable a payment process to occur prior to the rental of a portable vending machine or kiosk, during use of the portable vending machine or kiosk and/or after use of the portable vending machine or kiosk. In addition, traditional vending machines are not configured to enable payment for cleaning of the kiosk and/or items within the kiosk after use of the items. Thus, known kiosks have no provisions for paying a party for cleaning the kiosk and/or items within the kiosk, which may for example differ from the payee that is paid for the use of the items.

For at least the limitations described above there are no known wireless vending machines that are configured as portable point-of-sale vending machine to enable a user to dispense one or more reusable and interchangeable mechanical items such at least one mechanical product relating to pain relief, alternative health, fitness, postural alignment therapy, child care, elderly care, and at least one information guide and instructions relating to the at least one mechanical product. In addition, for at least the limitations described above, there are no known portable wireless point-of-sale vending machines that also enable a user to use the portable wireless point-of-sale vending machine as an exercise device, integrated service device, carrying device, baby carriage, or a physical therapy device in addition to the dispensation of one or more mechanical items.

### BRIEF SUMMARY OF THE INVENTION

One or more embodiments described in the specification are related to portable wireless point-of-sale vending machines that is easily moved and/or carried by a user, or which are self-propelled, and includes an outer surface and at least one inner portion enclosed by the outer surface. The one or more inner portions include one or more receptacles, wherein each of the receptacles contains one or more reusable and interchangeable mechanical items, for example exercise devices, medical devices or physical therapy devices. In at least one embodiment of the invention, the one or more mechanical items may also be depletable items, such that a supply level of the mechanical product is consumed and is able to be resupplied. According to one or more embodiments of the invention, the mechanical items are dispensed out of the outer surface as needed and the portable wireless point-of-sale vending machine may also be structurally used as an exercise device, integrated service device, carrying device, baby carriage or physical therapy device. In one or more embodiments of the invention, the portable wireless point-of-sale vending machine delivers goods and services, processes payments and discourages theft. In addition, in one or more embodiments, the vending machine has at least three modes of operation. For example, the modes of operation may include vending products or services, being locked down to protect the products and mechanical items, and being serviced by designated personnel or third parties such as housekeeping or other persons or logic machines.

Embodiments of the invention may be implemented with a wireless transceiver to communicate in a bidirectional manner with one or more third parties, and an embedded or removable computer that also communicate in a bidirectional manner with one or more third parties via the wireless transceiver. The wireless transceiver may include an RFID reader. According to embodiments of the invention, the embedded or removable computer automatically monitors usage and consumption of the vending machine and one or more reusable and interchangeable mechanical items. One or more embodiments of the invention may also include features on the outer surface of the vending machine such as at least one user-adjustable feature and a locking and unlocking mechanism.

According to at least one embodiment of the invention, the outer surface comprises a handle and an openable top surface, wherein the openable top surface includes an exterior padding. The top surface is openable to allow the mechanical items to be accessed from an inner portion of the at least one inner portion from the top surface when the top surface is opened. The openable top surface may remain connected to the outer surface while open. Furthermore, in one or more embodiments of the invention, the vending machine structurally includes one or more of at least two legs, a base, a drawer sliding along the base, at least two wheels, at least two extensions visible from the outside and either directly or indirectly connected to the outer surface. One or more embodiments may also include shape extensions, such that the shape extensions and the user-adjustable features may include one or more of the two legs, the base, the drawer sliding along the base, the at least two wheels, and the at least two extensions. Other self-propelled embodiments may include at least one motor driven wheel or track and power source to drive the motor for example.

Embodiments of the invention allow the embedded or removable computer to communicate with the locking and unlocking mechanism in order to lock and unlock the portable wireless point-of-sale vending machine based on input information from the user. Furthermore, in one or more embodiments, the embedded or removable computer manages inventory supplies of the one or more reusable and interchangeable mechanical items and communicates inventory information to the one or more third parties. In addition, the embedded or removable computer may electronically accept payment information of the user when the one or more reusable and interchangeable mechanical items are dispensed. The embedded or removable computer may accept payment from the one or more third parties, or directly from the user using the portable wireless point-of-sale vending machine, or any combination thereof. Also, in one or more embodiments, the embedded or removable computer communicates maintenance information and/or payment to the one or more third parties regarding the portable wireless point-of-sale vending machine.

By way of one or more embodiments, the portable wireless point-of-sale vending machine is in itself an exercise device, integrated service device, carrying device, baby carriage or a physical therapy device such as a postural alignment exercise device.

In at least one embodiment of the invention, the one or more reusable and interchangeable mechanical items are at least two different items, and include at least one mechanical product relating to pain relief, alternative health, fitness, postural alignment therapy and at least one information guide and instructions relating to the at least one mechanical product.

According to one or more embodiment, the portable wireless point-of-sale vending machine may be implemented with a screen, a touch screen, a computing device such as tablet computer, mobile device, an iPad®, a GPS device, and a clip for holding any one or more elements of the machine previously mentioned and mentioned hereafter. The screen and computing device may allow for communication with mobile devices, cell phones, television channels, the Internet, networks, circuits, radio stations, satellites and any other related communication and computing element. In at least one embodiment, the embedded or removable computer monitors user usage and consumption to output marketing and advertising information to the user on the screen, iPad®, a GPS device or any of the previously mentioned computing and communication elements.

According to one or more embodiment, the portable wireless point-of-sale vending machine includes a power source of at least one of on-board batteries and a retractable power cord which charges the on-board batteries and provides power to the vending machine.

### BRIEF DESCRIPTION OF THE DRAWINGS

The above and other aspects, features and advantages of the invention will be more apparent from the following more particular description thereof, presented in conjunction with the following drawings wherein:

FIG. 1 shows a front-view of the portable wireless point-of-sale vending machine according to one or more embodiments of the invention.

FIG. 2 shows a side-view of the portable wireless point-of-sale vending machine according to one or more embodiments of the invention.

FIG. 3 shows a top view of the inner portion and receptacles thereof of the portable wireless point-of-sale vending machine according to one or more embodiments of the invention.

FIG. 4 shows an overall diagram of the portable wireless point-of-sale vending machine used as a physical therapy apparatus according to another one or more embodiments of the invention.

### DETAILED DESCRIPTION OF THE INVENTION

A portable wireless point-of-sale vending machine for dispensing one or more reusable and interchangeable mechanical items and used as at least one of an exercise device, integrated service device, carrying device, baby carriage or a physical therapy device will now be described. In the following exemplary description numerous specific details are set forth in order to provide a more thorough understanding of embodiments of the invention. It will be apparent, however, to an artisan of ordinary skill that the present invention may be practiced without incorporating all aspects of the specific details described herein. In other instances, specific features, quantities, or measurements well known to those of ordinary skill in the art have not been described in detail so as not to obscure the invention. Readers should note that although examples of the invention are set forth herein, the claims, and the full scope of any equivalents, are what define the metes and bounds of the invention.

FIG. 1 illustrates a front-view of the portable wireless point-of-sale vending machine according to one or more embodiments of the invention. Specifically, FIG. 1 depicts a portable wireless point-of-sale vending machine 100 that is easily moved and/or carried by a user. The apparatus includes an outer surface defined by the exterior surface of the portable wireless point-of sale vending machine 100. One or more embodiments include an openable top surface 101, legs 102, wheel 103, handle 104, extension 105, base 106, and at least one inner portion enclosed by the outer surface (shown in FIG. 2). In one or more embodiments of the invention, the openable top surface 101 includes exterior padding. The top surface is openable to allow the mechanical items to be accessed from an inner portion of the at least one inner portion from the top surface when the top surface is opened, and in one or more embodiments remains connected to the outer surface while open.

The one or more inner portions include one or more receptacles, where each of the receptacles contains one or more reusable and interchangeable mechanical items (shown in FIG. 3). According to one or more embodiments of the invention, the mechanical items are dispensed from under or out of the openable top surface 101 as needed. The mechanical items may be dispensed from any moveable or openable element of the outer surface. For example, as will be discussed further, some of the moveable or openable elements include the padding on the openable top surface 101, the openable top surface 101 itself and a drawer 203 as shown in FIG. 2. In addition, one or more embodiments of the invention may utilize any combination or numbers of wheels 103 and extensions 105 at the bottom of the vending machine 100. The wheels enable the vending machine 100 to be easily moved and transported by the user from one location to another. Handle element 104 allows for the user to pull or position the vending machine 100 for example.

Furthermore, the entire portable wireless point-of-sale vending machine 100 may also be structurally used as an exercise device, integrated service device, carrying device, baby carriage or physical therapy device. For example a user may utilize padding on openable top surface 101 for example to rest any part of the user's body and undergo or perform physical therapy or other type of activity. In at least one embodiment of the invention, the vending machine 100 is used as a physical therapy device allowing the user to place his or her legs on padding on openable top surface 101 while lying down on the ground, for example at an angle with his or her back on the ground as is shown in FIG. 4. The extensions 105 may be utilized to set the height of the apparatus or level the apparatus. For example, the extensions enable the user to set the height of the openable top surface with respect to the floor as desired to obtain a preferred height for physical therapy.

Embodiments of the invention may be implemented with a wireless transceiver to communicate in a bidirectional manner with one or more third parties 150 and optionally 151. Embodiments may include one or more embedded or removable computers that also communicate in a bidirectional manner with one or more third parties via the wireless transceiver. In at least one embodiment of the invention, the wireless transceiver may include or couple with an RFID reader. The one or more third parties 150 and 151 range from logic machines, hotel personnel, distribution companies, networks, storage devices, distributed databases, banks, pharmacies, computer systems that accept payment, etc. For example, according to one or more embodiments, when the vending machine is used in a hotel environment, the user is able to rent the entire portable wireless point-of-sale vending machine 100 to use in multiple locations, with unlimited access to the products and mechanical items within the vending machine 100 for a limited amount of time. The limited amount of time is determined by the user at the time of rent, or determined by designated personnel at the location of the vending machine 100 (e.g. hotel). Furthermore, according to one or more embodiments of the invention, the vending machine 100 is available to the user if the user is at the rental location of the vending machine 100, or the vending machine is delivered to the location of the user either manually or electronically. When delivered manually, the vending machine 100 is delivered to the user via designated personnel in the vicinity of the vending machine (e.g. hotel). When delivered electronically, the vending machine 100 is delivered to the user via a mechanical device, and/or via self-navigation robotic commands embedded within the vending machine using the one or more embedded or removable computers of the vending machine and/or via communication of navigational robotic commands from the one or more third parties to the vending machine 100. The robotic commands are either programmed into the embedded or removable computers of the vending machine 100, or are communicated wirelessly to the vending machine via the wireless transceiver. In this embodiment, extensions 105 may couple with one or more drive wheels for example so that the apparatus may move on four wheels for example.

According to embodiments of the invention, the embedded or removable computer automatically monitors usage and consumption of the vending machine 100, as well as monitors usage and consumption of the one or more reusable and interchangeable mechanical items. In other embodiments, the removable computer may be owned by the user and interface with the apparatus wirelessly as one skilled in the art will appreciate. One or more embodiments of the invention may also include features on the outer surface or accessible from outside of the vending machine 100 such as at least one user-adjustable feature and a locking and unlocking mechanism. The locking and unlocking mechanism includes one or more of key locks, code locks, card readers, computer controlled locks, a touch screen and any combination thereof. In one or more embodiments, the locking and unlocking mechanism may be operated by the embedded or removable computer and/or by a controlling computer located away from the vending machine 100 to enforce limited access to the vending machine. According to one or more embodiments, the locking and unlocking mechanism is controlled using a manual and/or computer controlled authentication mechanism necessary to change the state of the vending machine from a lockdown state to a vending state. In addition, according to one or more embodiments, the portable wireless point-of-sale vending machine 100 may be implemented with a rolling and/or carrying case enclosing the outer surface, for easy of portability and transportation. Furthermore, in at least one embodiment, the case itself may also enter into a self-protective lockdown state for security and privacy using a locking mechanism. The locking mechanism may include any one or more of the locking and unlocking mechanisms discussed previously regarding the locking and unlocking mechanism of the vending machine 100. Furthermore, in at least one embodiment of the invention, the case and/or the portable wireless point-of-sale vending machine 100 may remain stationary in a standard location, or may be transported to the user upon request. According to one or more embodiments, the case and/or the vending machine 100 itself is able to stack with other devices, and able to stack several mechanical items in at least one or more of the receptacles for efficient storage.

FIG. 2 illustrates a side-view of the portable wireless point-of-sale vending machine according to one or more embodiments of the invention. Specifically, FIG. 2 depicts the vending machine 100 with the at least two legs 102, base 106, drawer 203 sliding along the base 106, with extensions 105 visible from the outside and either directly or indirectly connected to the outer surface. The drawer may hold exercise devices, physical therapy devices, a computing element or any other item for example. In addition, the drawer may be lockable so that it does not open unless authorized to do so. In one or more embodiments, the drawer includes cutouts that enable items to rest within the cutouts wherein the items are optionally configured to not be removed without extending the drawer. One or more embodiments may also include shape extensions, such that the shape extensions and the user-adjustable features may include one or more of the two legs 102, the drawer 203 sliding along the base 106, the at least two wheels 103 and the at least two extensions 105. In addition, according to one or more embodiments, the user-adjustable features and shape extensions are adjusted to allow the vending machine 100 to accommodate different exercises and physical therapy techniques for users of different heights and weights. For example, the user-adjustable legs 102 may be lengthened or shortened, and shape extensions may be added, removed, or adjusted to increase or decrease the height, width and angle of the vending machine 100 as desired. Embodiments of wheels 103 may also include height-adjusting elements if desired, for example an extension element as utilized for extensions 105 that couples with one or more axles that couple to each wheels as one skilled in the art will recognize.

FIG. 3 illustrates a top view of the inner portion and receptacles thereof of the portable wireless point-of-sale vending machine according to one or more embodiments of the invention. Information display 301 may include instructional or advertising information for example and is viewable after openable top surface 101 is opened, and which further enables access to the other components stored within the apparatus. The one or more inner portions include one or more receptacles, wherein each of the receptacles contains the one or more reusable and interchangeable mechanical items. For example, receptacles 302-304 may hold a heating element, massage device, exercise or sanitary mat respectively and a gap may exist between information display 301 and the top of the receptacles to hold a pad or mat or for example needle-less acupuncture element 308. Embodiments may include receptacles or objects such as informational brochure 305, computing element 306, battery and/or power cord 307 or any other element respectively for example. Receptacles 309-311 may hold exercise blocks, pads or computing elements or any combination thereof or any other items for example. Optional locking mechanism 320 may include any type of locking mechanism previously mentioned. Computing element 306 may include a wireless transceiver that is able to communicate wirelessly with third party 150 and/or optionally third party 151 for example to pay for use of the apparatus, and optionally to pay for cleaning the apparatus and/or components therein. In one or more embodiments, the payment information may travel through one external computer or two or more external computers to pay the same or different entities. In at least one embodiment of the invention, the one or more reusable and interchangeable mechanical items are at least two different items, and include at least one mechanical product relating to pain relief, alternative health, fitness, postural alignment therapy and at least one information guide and instructions relating to the at least one mechanical product. In at least one embodiment of the invention, the one or more mechanical items may also be depletable items, such that a supply level of the mechanical product is consumed and is able to be resupplied. One or more embodiments of the invention may be utilized to store any type of medical device, including but not limited to a continuous positive airway pressure (CPAP) device, for example to treat sleep apnea, or oxygen for persons with respiratory deficiencies. Other examples of devices may include magnification devices for viewing or reading or illumination devices, devices to aid those with disabilities, or to comply with any disability guidelines, such as the American Disabilities Act (ADA), or any combination thereof.

Embodiments of the invention allow the embedded or removable computer to communicate with the locking and unlocking mechanism 320 in order to lock and unlock the portable wireless point-of-sale vending machine 100 based on input information from the user. Furthermore, in one or more embodiments, the embedded or removable computer manages inventory supplies of the one or more reusable and interchangeable mechanical items via the RFID reader, for example that is coupled with computing element 306 and communicates inventory information to the one or more third parties. In one or more embodiments, during the limited rental time, the vending machine is fully owned by the user, in which the user is able to monitor inventory and re-order supplies, products and/or mechanical items as deemed necessary.

In addition, the embedded or removable computer according to one or more embodiments may electronically accept payment information of the user before, during or after the one or more reusable and interchangeable mechanical items are dispended or dispensed. In one or more embodiments, the payment information is accepted from the one or more third parties and/or directly from the user using a payment process mechanism located on or within the portable wireless point-of-sale vending machine 100. The different payment process mechanisms may include using one or more of a credit card reader, a cash collector, a coin collector, a non-credit card reader, a keypad, a touch screen and a voice recognizer. One skilled in the art will recognize that these components may be directly coupled with computing element 306 or indirectly coupled with computing element 360, e.g., as standalone elements for example. Such direct manual payment process mechanisms provides the user with a variety of mechanisms to rent the vending machine 100 and the mechanism items placed within the vending machine. The payment process mechanisms may or may not communicate with the third parties, depending on the type of payment process mechanism. For example, if the user is charged for usage and consumption after using the vending machine, then the embedded or removable computer and/or the user may directly and/or automatically communicate with the third party which would charge a user via a stored and recognized payment process that was established prior to the renting of the vending machine, e.g., third party 150. In addition to or alternatively, one or more embodiments allow the user to manually apply a payment process mechanism without the need to communicate with the one or more third parties. In addition, one or more embodiments allow the embedded or removable computer to automatically communicate with a third party and charge a user's account, credit card, or other stored payment process mechanism in case of theft and unpaid invoices without the need to query the user or obtain authentication from the user. Another third party 151 may be paid for cleaning of the apparatus or items within the apparatus as previously discussed.

In one or more embodiments, the portable wireless point-of-sale vending machine may be rented out, charged, and/or used for dispensation on a weekly basis, an hour basis, a per-night basis or any other time limited basis. In addition, according to one or more embodiment of the invention, the user is able to extend the rental period using one or more of the embedded or removable computers, which may include a touch screen, or other screen by checking to see if a time slot is available for the desired rental period. Time slot availability is recognized via communication between the wireless transceiver and one or more of the third parties, and/or via communication between the wireless transceiver and a data storage or network device.

Also, in one or more embodiments, the embedded or removable computer communicates maintenance information to the one or more third parties regarding mechanical errors, computer errors, structural errors or routine maintenance of the portable wireless point-of-sale vending machine 100. The one or more third parties, according to at least one embodiment of the invention, include one or more of information technology, hotel housekeeping, maintenance personnel located in the vicinity of the vending machine, other managerial designated personnel with access to the vending machine, and distributed databases or networks with storage capabilities. The distributed databases or networks with storage capabilities may be located around the vicinity of the vending machine or may be located remotely, such as at a location out the hotel, in a cloud computing environment, in a different city, in a different state or elsewhere.

In at least one embodiment of the invention, the one or more reusable and interchangeable mechanical items are at least two different items, and include at least one mechanical product relating to pain relief, alternative health, fitness, postural alignment therapy and at least one information guide and instructions relating to the at least one mechanical product. In at least one embodiment of the invention, the one or more mechanical items may also be depletable items, such that a supply level of the mechanical product is consumed and is able to be resupplied. In addition, in at least one embodiment of the invention, the one or more reusable and interchangeable mechanical items may include supplemental and instructional media on multiple channels such as one or more of a website and a closed circuit television.

According to one or more embodiment, the portable wireless point-of-sale vending machine 100 may be implemented with a screen, a touch screen, a computing device 306 such as an iPad®, or any other type of portable computer, and may internally include or externally couple with a GPS device, and a clip for holding any one or more elements of the machine previously mentioned and mentioned hereafter. The screen and computing device may allow for communication with mobile devices, cell phones, television channels, the Internet, networks, circuits, radio stations, satellites and any other related communication and computing element. In at least one embodiment, the embedded or removable computer monitors user usage and consumption to output marketing and advertising information to the user on the screen, iPad®, a GPS device or any of the previously mentioned computing and communication elements. Embodiments of the invention may use the GPS device for location identification in order to easily and efficiently monitor the location of the vending machine 100 for security, management, inventory, maintenance, theft, misplacement and cleaning purposes. Furthermore, in one or more embodiments of the invention, the embedded or removable computer allows for automatic receipt transmission to the user, and further allows for marketing and loyalty programs. The embedded or removable computer may use the user's demographic information, the user's rental history in terms of usage and consumption, as well as user's rental habits for further marketing and advertising strategies specialized for the specific user during the time of rental based on the acquired information. In addition, one or more embodiments of the invention may use the one or more embedded or removable computers, touch screen, wireless transceiver, or any other element of the vending machine 100 and any combination thereof for communication with an online store that sells the one or more reusable and interchangeable mechanical items or similar mechanical items allowing the user to purchase a desired mechanical item. In one or more embodiments, the one or more embedded or removable computers may alert and query the user for a payment process to either automatically charge the user based user payment process information previously stored in the vending machine or previously stored with the one or more third parties, or to transfer an invoice or bill to the one or more third parties for a payment process mechanism after the expiry of the rental period, or to allow the user for a direct payment to the vending machine using the one or more previously discussed payment process mechanisms (e.g. a credit card reader, a cash collector, a coin collector, a non-credit card reader, a keypad, a touch screen, a voice recognizer, etc.).

According to one or more embodiment, the portable wireless point-of-sale vending machine 100 may utilize any type of power source coupled with the computing element 306 as previously discussed or that includes any combination of on-board batteries, a retractable power cord and a power strip, solar panel or inductive charger, or any other power source or generating device, which are not shown for brevity, which alone or in combination charge the vending machine and provide power to the on-board batteries. In addition, in one or more embodiments of the invention, the vending machine 100 may be wirelessly powered using one or more wireless chargers in the vicinity of the vending machine. In other embodiments, solar or inductive charging may be utilized to provide power to the apparatus.

FIG. 4 illustrates a perspective view of the portable wireless point-of-sale vending machine during use as a physical therapy apparatus according to another one or more embodiments of the invention. According to one or more embodiments of the invention, the one or more reusable and interchangeable mechanical items include wellness regimen informational guides and instructions explaining how the outer surface and/or the case may be used a physical therapy apparatus and for postural alignment therapy as previously discussed.

While the invention herein disclosed has been described by means of specific embodiments and applications thereof, numerous modifications and variations could be made thereto by those skilled in the art without departing from the scope of the invention set forth in the claims.

What is claimed is:

1. A portable wireless point-of-sale vending machine comprising:
an outer surface and at least one inner portion enclosed by said outer surface;
one or more receptacles located within at least one of said at least one inner portion;
one or more reusable and interchangeable mechanical items located in each of said one or more receptacles respectively and configured to be dispensed out of said outer surface;
a wireless transceiver configured to communicate with one or more third parties;
a computer coupled with said wireless transceiver;
at least one user-adjustable feature located on said outer surface;
wherein said portable wireless point-of-sale vending machine is configured to be easily moved and carried by a user;
wherein said computer is configured to dispense and automatically monitor usage and consumption of at least one of said portable wireless point-of-sale vending machine and said one or more reusable and interchangeable mechanical items, and
wherein said portable wireless point-of-sale vending machine is configured to be used as an exercise device or physical therapy device.

2. The portable wireless point-of-sale vending machine of claim 1, wherein said outer surface comprises a handle.

3. The portable wireless point-of-sale vending machine of claim 1, wherein said outer surface comprises an openable top surface, wherein said openable top surface comprises an exterior padding.

4. The portable wireless point-of-sale vending machine of claim 1, wherein said outer surface comprises wheels.

5. The portable wireless point-of-sale vending machine of claim 1, further comprising a locking and unlocking mechanism located on said outer surface and wherein said computer is in communication with said locking and unlocking mechanism such that said computer is further configured to lock and unlock said portable wireless point-of-sale vending machine based on input information from said user or from remote authorization services.

6. The portable wireless point-of-sale vending machine of claim 1, wherein said computer is further configured to manage inventory supplies of said one or more reusable and interchangeable mechanical items and communicate inventory information to said one or more third parties.

7. The portable wireless point-of-sale vending machine of claim 1, wherein said computer is further configured to electronically accept payment information of said user when said one or more reusable and interchangeable mechanical items are dispensed.

8. The portable wireless point-of-sale vending machine of claim 7, wherein said computer configured to electronically accept payment information of said user comprises electronic transfer of payment to said one or more third parties.

9. The portable wireless point-of-sale vending machine of claim 7, wherein said computer configured to electronically accept payment information of said user comprises electronic acceptance of payment directly from said user via said portable wireless point-of-sale vending machine.

10. The portable wireless point-of-sale vending machine of claim 1, wherein said computer is further configured to communicate maintenance information to said one or more third parties regarding said portable wireless point-of-sale vending machine.

11. The portable wireless point-of-sale vending machine of claim 1, wherein said computer comprises an RFID reader.

12. The portable wireless point-of-sale vending machine of claim 1, wherein said at least one user-adjustable feature comprises at least one of adjustable legs and adjustable integrated shape extensions.

13. The portable wireless point-of-sale vending machine of claim 1, wherein said portable wireless point-of-sale vending machine configured as an exercise device or physical therapy device comprises a postural alignment exercise device.

14. The portable wireless point-of-sale vending machine of claim 1, wherein said one or more reusable and interchangeable mechanical items comprises at least two different items.

15. The portable wireless point-of-sale vending machine of claim 1, wherein said one or more reusable and interchangeable mechanical items comprises at least one mechanical product relating to exercise, fitness, medical, physical therapy, pain relief, alternative health, postural alignment therapy and at least one information guide relating to said at least one mechanical product.

16. The portable wireless point-of-sale vending machine of claim 1, further comprising a screen, and wherein said computer is further configured to use said monitored user usage and consumption to output marketing and advertising information to said user on said screen.

17. The portable wireless point-of-sale vending machine of claim 1, further comprising a power source.

18. The portable wireless point-of-sale vending machine of claim 15, wherein said power source comprises on-board batteries.

19. The portable wireless point-of-sale vending machine of claim 16, further comprising a retractable power cord or solar panel or inductive charger configured to charge said on-board batteries and power said portable wireless point-of-sale vending machine.

20. The portable wireless point-of-sale vending machine of claim 1, further comprising a touch screen.
